(19) United States
(12) Patent Application Publication
(10) Pub. No.: US 2010/0111890 A1
(43) Pub. Date: May 6, 2010

(54) USE IN PERFUMERY AND FLAVORING AND PROCESS FOR THE PREPARATION OF 5,5-DIMETHYL-3-ETHYL-3,4-DIHYDROFURAN-2-ONE

(75) Inventors: Jean Mane, Grasse (FR); Jean-Claude Clinet, Villeneuve-Loubet (FR); Isabel Clinet, Villeneuve-Loubet (FR); Lydie Coulombel, Mouans-Sartoux (FR); Christophe Martin, Nice (FR)

Correspondence Address: YOUNG & THOMPSON, 209 Madison Street, Suite 500, Alexandria, VA, 22314, US

(73) Assignee: V. MANE FILS, Bar Sur Loup (FR)

(21) Appl. No.: 12/444,224

(22) PCT Filed: Sep. 25, 2007
(86) PCT No.: PCT/FR07/52004

§ 371 Date: Apr. 3, 2009

(30) Foreign Application Priority Data
Oct. 3, 2006 (FR) 0608661

(51) Int. Cl. C07D 307/33; A61K 8/49; C11D 3/50; A61L 9/01; A23L 1/226

(52) U.S. Cl. 424/76.2; 512/11; 510/103; 514/772; 549/475; 426/536

(57) ABSTRACT

The invention relates to the use of 5,5-dimethyl-3-ethyl-3,4-dihydrofuran-2-one (A), named “Noxolide” by the applicant, as an olfactory agent in perfumery, and also as a food flavouring. The invention also relates to a novel process for the preparation of 5,5-dimethyl-3-ethyl-3,4-dihydrofuran-2-one.

The present invention relates to the use of 5,5-dimethyl-3-ethyl-3,4-dihydrofuran-2-one, known as “Noxolide” by the Applicant Company, as odorous agent in perfumery or food flavoring.

The invention also relates to a novel process for the preparation of 5,5-dimethyl-3-ethyl-3,4-dihydrofuran-2-one.

The terms “fragrance”, “fragrant” or “odorous” are used interchangeably here for any organoleptic compound which stimulates the sense of smell in a pleasing fashion.

The terms “flavor” and “flavoring” are used interchangeably here for any organoleptic compound which stimulates the taste.

The term “organoleptic compound” is used here for any compound which stimulates the senses of smell and taste and which is thus perceived as having a characteristic smell and/or taste.

The term “to mask” or “masking” is understood to mean reducing or removing the perception of an unpleasant smell or an unpleasant taste generated by one or more molecules participating in the composition of a product.

Odorous molecules belong to various chemical categories. Mention may in particular be made of lactones, which are widely distributed in the natural state and which constitute a family of odorous molecules of great importance in perfumery. Lactones are known for their fruity notes, such as, for example, the γ-nonanoic lactone (coconut fragrance) described in Bunce R. A. and Reeves H. D., J. Chem. Ed., 1990, 67, 69-70. Other well known fragrances of lactones are described with terms such as herbal, hay, spicy, woody, almondy, milky, sweet fruits, such as peach or apricot, and the like.

Lactones are present in the form of traces in fruits, milk products and alcoholic drinks but contribute substantially to their flavors (Duffosé L. et al., Sciences des Aliments, 1994, 14, 17-50). The low threshold for the olfactory detection of lactones is compensated for by the use of the latter in a large amount in the field of perfumes and flavors (Zope D. D. et al., Fafai Journal, 2004, 43-54).

Some lactones contribute the main note of a given flavor; this note is often pleasant and persistent. The Applicant Company has demonstrated, during its research, that 5,5-dimethyl-3-ethyl-3,4-dihydrofuran-2-one, belonging to the family of the trisubstituted γ-lactones, denoted under the name of “Noxolide”, exhibits such characteristics.

To the knowledge of the Applicant Company, 5,5-dimethyl-3-ethyl-3,4-dihydrofuran-2-one has been reported twice in the literature. First during a study on the effects of substituent in 1,2-hydride transfer reactions (Kirmse W. et al., Chem. Ber., 1975, 108, 1839 & de Campos M. et al., Arch. Pharm. (Weinheim), 1965, 298, 92). More recently, 5,5-dimethyl-3-ethyl-3,4-dihydrofuran-2-one has been described as one of the products of the reaction of butanoic acid with the “naphthalene/lithium” organometallic complex, followed by a condensation with isobutylene oxide (Fujita et al., J. Appl. Chem. Biotechnol., 1977, 27, 539).

However, none of the documents of the prior art describes or suggests that 5,5-dimethyl-3-ethyl-3,4-dihydrofuran-2-one has organoleptic properties and, a fortiori, the use which might be made of this molecule as odorous compound, in particular fragrance, or as flavor, or also as agent for masking a smell or a flavor, is neither described nor suggested.

Furthermore, the syntheses provided are difficult to apply on the industrial scale, involving in particular the use of alkali metals which are highly reactive with regard to water and oxygen of the air.

Thus, the technical problem which the Applicant Company intends to solve by the present invention is that of making available novel molecules which are highly odorous, which are stable, which have a long shelf life and which are prepared according to a simple method of preparation from inexpensive and plentiful starting materials.

The simplicity of implementation of the reactions and the low cost of the starting materials used represent significant advantages in the industrial use of the invention.

A subject matter of the present invention is the use of 5,5-dimethyl-3-ethyl-3,4-dihydrofuran-2-one (or Noxolide) as odorous agent or compound, in particular as fragrance or flavor. An evaluation panel, composed of several experts, qualitatively evaluated the compound of the invention. Noxolide, represented below by the formula (A), was described as marine, iodine with the following facets: nutty, anise slightly licorice, patchouli, damascenone.

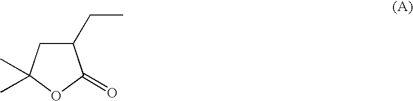

Due to its olfactory qualities, Noxolide finds very varied use in perfumery in the preparation of scenting compositions, scenting bases or concentrates, perfumes or eaux de toilette, and in the preparation of various scented articles, such as soaps, foam baths, shower or bath gels, aftershave lotions, shampoos or other hair products, in particular hair hygiene or hair care products, cosmetic preparations, deodorants for the body or for the surrounding air, or also textile detergents or softeners or products for the upkeep of or for purifying the surrounding air.

In particular, a subject matter of the invention is a hair hygiene or care product, characterized in that it comprises 5,5-dimethyl-3-ethyl-3,4-dihydrofuran-2-one.

In these applications, the compound according to the invention can be employed alone or, as is more common in perfumery, as a mixture with other scenting ingredients, solvents or adjuvants of common use in perfumery and which a person skilled in the art is in a position to choose according to the effect desired and the nature of the product to be scented.

Another subject matter of the present invention is the use of 5,5-dimethyl-3-ethyl-3,4-dihydrofuran-2-one (Noxolide) as flavor. Noxolide is of use as food flavor, particularly for the preparation of food compositions, food additives, flavoring bases and concentrates, and in the preparation of various flavored products, such as drinks, dairy products, ice creams, soups, sauces, ready-made meals, meat-based products, cooking aids, biscuits or salty snacks. Noxolide can also be used as flavor in wine, beer and/or tobacco.

Another subject matter of the present invention is the use of 5,5-dimethyl-3-ethyl-3,4-dihydrofuran-2-one (Noxolide) as masking agent for a taste and/or a smell, in particular in a food or cosmetic or pharmaceutical composition.

In these applications, the compound of the invention can be employed alone or, as is more common in flavorings, as a mixture with other flavoring ingredients, solvents or adjuvants of common use in food flavoring and which a person skilled in the art is in a position to choose according to the effect desired and the nature of the product to be flavored.

The concentrations under which 5,5-dimethyl-3-ethyl-3,4-dihydrofuran-2-one (Noxolide) can be used in order to obtain the desired scenting, flavoring and/or masking effects vary in a very wide range of values from 0.001% to 99% by weight, preferably from 0.001% to 20% by weight and very preferably from 0.1% to 10% by weight, it being well known that these values depend on the nature of the article to be scented, on the odorous effect desired and on the nature of the other ingredients in a given composition.

Another subject matter of the invention is a novel process for the synthesis of 5,5-dimethyl-3-ethyl-3,4-dihydrofuran-2-one (Noxolide) comprising
a first stage [1] in which sodium methoxide and dimethylmalonate are reacted with 3-chloro-2-methylprop-1-ene, resulting in the formation of 4-methyl-2-(methoxycarbonyl)pent-4-enoate (I),
a second stage [2] in which the product (I) is reacted with potassium t-butoxide and ethyl bromide, resulting in the formation of 2-ethyl-4-methyl-2-(methoxycarbonyl)pent-4-enoate (II),
a third stage [3] in which the product (II) is reacted with sodium hydroxide and phosphoric acid, resulting in the formation of 5,5-dimethyl-3-ethyl-3,4-dihydrofuran-2-one (A).

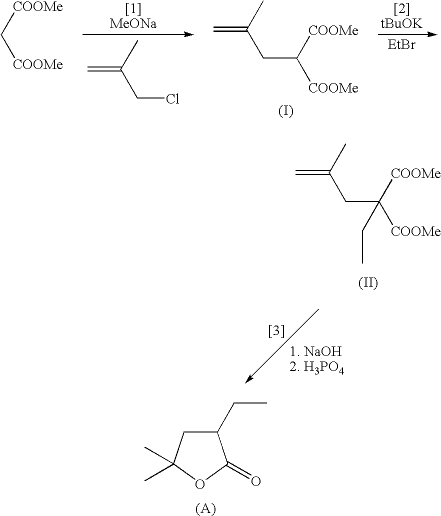

The following examples further illustrate the various processes for the manufacture of the novel compounds according to the invention, and also their use and their advantage. These examples are presented only for the purpose of illustration and may not be regarded as limiting the invention.

### EXAMPLE 1

### Production of Noxolide or 5,5-dimethyl-3-ethyl-3,4-dihydrofuran-2-one

### Methyl 4-methyl-2-(methoxycarbonyl)pent-4-enoate (I)

The reaction is carried out in a 1 l three-necked round-bottomed flask equipped with a thermometer dipping into the reaction medium, an upright reflux condenser equipped with a potassium hydroxide drying tube and a 250 ml pressure-equalizing dropping funnel.

54 g of sodium methoxide, 300 ml of anhydrous methanol, 1 g potassium iodide and then 138.7 g of dimethyl-malonate are successively introduced into the dry round-bottomed flask purged with nitrogen. The resulting solution is stirred at ambient temperature for 1 hour and then 95 g of 3-chloro-2-methylprop-1-ene are slowly added from the dropping funnel. The mixture is stirred at ambient temperature for 3 hours and then at reflux for 12 hours. The reflux condenser is replaced with a distillation assembly and the solvent is distilled off. The residue is taken up in 50 ml of clear water and 300 ml of methylcyclohexane. The organic phase is dried and then the solvent is removed by distillation. The residue is fractionated using a 30 cm Vigreaux column; 3 fractions are collected:
The first fraction, which weighs 15 g, is composed of dimethylmalonate (B.p.=85-87° C./20 mmHg)
The second fraction weighs 138 g and ends at 86° C./6 mmHg. It is composed of the desired ester (I), i.e. a yield of 74% (80% with respect to the starting material actually consumed). The product is more than 98% pure by Gas Chromatography.
A third fraction is obtained by high vacuum distillation (B.p.=64-68° C./1 mmHg). It weighs 36 g and has been identified as dimethyl 2,2-bis(2-methyl-2-propenyl)malonate.

### For the Compound I:

IR: (NaCl, film) 1760, 1740, 1440, 1350, 1295, 1230, 1150, 1050, 1020, 900 cm⁻¹

¹H NMR, (CDCl₃)

1.73 ppm (m, 3H); 2.50 (d, J=8 Hz, 2H); 3.20 to 3.70 (m, H); 3.68 (s, 6H); 4.72 (m, 2H).

MS: 70 eV

186 (M^{+.}); 154; 127; 111; 95 amu

### Methyl 2-ethyl-4-methyl-2-(methoxycarbonyl)pent-4-enoate (II)

The reaction is carried out in a 1 l three-necked round-bottomed flask equipped with a thermometer dipped into the reaction medium, an upright reflux condenser equipped with a potassium hydroxide drying tube and a 250 ml pressure-equalizing dropping funnel.

93 g of methyl 4-methyl-2-(methoxycarbonyl)pent-4-enoate (I) and 40 g of anhydrous DMSO are successively introduced into the round-bottomed flask. 59 g of potassium t-butoxide are added, the mixture being maintained between 20 and 30° C. using a cooling bath, and that mixture obtained is stirred at ambient temperature for 2 hours before 35.5 g of ethyl bromide are slowly added to the round-bottomed flask via the dropping funnel, so that the temperature does not exceed 30° C. in the medium. The solution obtained is stirred at ambient temperature for 12 hours and then run onto 300 ml of 5% hydrochloric acid. The resulting aqueous phase is extracted 5 times with 100 ml of methylcyclohexane. The combined organic phases are washed with 100 ml of clear water and then dried and concentrated under vacuum. The residue is rectified under vacuum. The product distils at 95° C./4 mmHg. 91 g of desired product are isolated, i.e. a yield of 85%.

¹H NMR, (CDCl₃)

0.64 ppm (t, 7.5 Hz, 3H); 1.06 (t, 7.1 Hz, 6H); 1.46 (s, 3H); 1.75 (q, 7.5 Hz, 2H); 2.50 (s, 2H); 4.00 (q, 7.1 Hz, 4H); 4.53 (s, 1H); 4.65 (s, 1H).

IR: (NaCl, film) 3079, 2955, 1736, 1647, 1437, 1301, 1225, 1123, 901 cm⁻¹

MS: 214 (M^{+.}); 185; 155; 139; 123; 95; 85; 79; 67 amu

### 5,5-Dimethyl-3-ethyl-3,4-dihydrofuran-2-one

The reaction is carried out in a 1 l three-necked round-bottomed flask equipped with a thermometer dipping into the reaction medium and an upright reflux condenser. The latter will subsequently be replaced with a conventional distillation system: Vigreaux column, condenser and receiver.

200 ml of clear water, 200 ml of methanol, 20 g of sodium hydroxide pellets and then 53.5 g of methyl 2-ethyl-4-methyl-2-(methoxycarbonyl)pent-4-enoate (II) are successively introduced into the round-bottomed flask. The mixture is brought to reflux for 4 hours and then the heating is interrupted. The distillation assembly replaces the upright reflux condenser and the solvent is distilled off until a temperature of 90° C. is obtained in the vapors. Hot water is introduced into the round-bottomed flask as the distillation proceeds in order to keep the distillation mixture fluid. The aqueous phase is brought to 60° C., it is extracted with 2 times 100 ml of toluene and then it is brought to between 0 and 5° C. A heavy white precipitate is formed. The suspension is acidified to pH=3 with a 6N sulfuric acid solution while keeping the temperature below 5° C. The white precipitate is filtered off, washed with clear water to neutrality and then taken up in 300 ml of dry toluene and brought to reflux in the presence of 1 g of 85% phosphoric acid for 12 hours.

The solution is cooled, washed to neutrality with a saturated sodium bicarbonate solution and then dried. The solvent is distilled off under partial vacuum and then the residue is rectified under vacuum (B.p.=86° C./6 mmHg). The lactone weighs 27.8 g, which represents a yield of 78.2%.

It has a strong smell of green walnuts. It is called Noxolide (Formula A).

¹H NMR, (CDCl₃)

1.0 ppm (t, 7.4 Hz, 3H); 1.4 (s, 3H); 1.5 (s, 3H); 1.4 to 1.6 (m, 1H); 1.7 (m, 1H); 1.8 to 2.0 (m, 1H); 2.3 (dd, 12.6/8.9 Hz, 1H); 2.7 (ddt, 11.2/8.9/4.7 Hz, 1H)

IR: (NaCl, film) 2973, 2935, 2878, 1768, 1461, 1376, 1271, 1168, 1118, 952 cm⁻¹

MS: (70 eV) 142 (M^{+.}); 127; 99; 83; 69 amu

¹³C NMR: CDCl₃, 50 MHz

12.0, 24.1, 27.5, 29.3, 41.0, 42.4, 82.5, 178.5 ppm

### Elemental Analyses:

| | | |
|---|---|---|
| % calc. | C 67.57 | H 9.92 |
| % found | C 66.84 | H 10.02 |

### Physical Constants:

n₂₀=1.4355

### D₂₀=0.949

### EXAMPLE 2

### Olfactory Evaluation of Noxolide in a Composition

Noxolide has been described in olfactory terms as marine, iodine with the following facets, nutty, anise slightly licorice, patchouli, damascenone.

A scenting composition was created (test 2) in which the olfactory impact of Noxolide was examined in comparison with a composition not comprising the compound (test 1).

| | Test 1 | Test 2 |
|---|---|---|
| Components | (weight) | (weight) |
| HS Melon P-99 | 10.00 | 10.00 |
| Bergamot 77 P-99 | 15.00 | 15.00 |
| Linalyl acetate HLR | 50.00 | 50.00 |
| Cassis Base 345 B | 5.00 | 5.00 |
| Lemon Terpenes USA | 50.00 | 50.00 |
| Dihydromyrcenol | 50.00 | 50.00 |
| Ethyl Linalol (Givaudan) | 90.00 | 90.00 |
| Floralozone (IFF) | 10.00 | 10.00 |
| Galaxolide without EP in IPM | 135.00 | 135.00 |
| Methyl dihydrojasmonate | 200.00 | 200.00 |
| Octahydrotetramethyl Acetonaphthone | 40.00 | 40.00 |
| Lilial (Givaudan) from MFI | 100.00 | 100.00 |
| Linalol HLR | 50.00 | 50.00 |
| γ-Nonalactone-Prunolide | 50.00 | 50.00 |
| C14 Aldehyde, γ-Undecalactone (10% | 5.00 | 5.00 |
| DPG) | | |
| Dihydro Floriffone (IFF) (10% DPG) | 5.00 | 5.00 |
| Folione (Givaudan) (1% DPG) | 20.00 | 20.00 |
| Jasmolactone (Firmenich) (10% DPG) | 5.00 | 5.00 |
| Triplal (IFF) (10% DPG) | 5.00 | 5.00 |
| Vanillin (Rhône-Poulenc) (10% DPG) | 5.00 | 5.00 |
| DPG | 2.70 | 0.00 |
| Noxolide | 0.00 | 2.70 |

The preceding compositions were tested in a shampoo application of 2 in 1 type in a proportion of 0.5% in the base.

The formulation of test 2 gives greater strength and response to the top note. The greener and more natural appearance than that of the formulation of test 1 is noteworthy. Noxolide particularly improves the transparent natural aqueous green side of the perfume accord. It emphasizes the sparkling citrus tonalities and the fruity lactonic accord. The formulation of test 1 is soft, musky, flat and without depth. It masks the base with difficulty.

1-10. (canceled)

11. A composition comprising 5,5-dimethyl-3-ethyl-3,4-dihydrofuran-2-one as an odorous agent providing a desired flavor or fragrance to said composition.

12. The composition of claim 11, wherein said composition is a food composition, and said 5,5-dimethyl-3-ethyl-3,4-dihydrofuran-2-one provides a food flavor.

13. The composition of claim 11, wherein said composition is selected from the group consisting of wine, beer and tobacco, and said 5,5-dimethyl-3-ethyl-3,4-dihydrofuran-2-one provides a flavor to said composition.

14. The composition according to claim 11, wherein said composition is selected from the group consisting of a food composition, a food additive, a flavoring base and flavoring concentrate.

15. The composition according to claim 14, wherein said composition is a food composition selected from the group consisting of a drink, a dairy product, an ice cream, a soup, a sauce, a ready-made meal, a meat-based product, a cooking aid, a biscuit and a salty snack.

16. The composition according to claim 11, wherein said composition is selected from group consisting of a scenting composition, a scenting base or concentrate, a perfume or eau de toilette, a soap, a foam bath, a shower or bath gel, aftershave lotion, a cosmetic preparation, a deodorant for the body or for the surrounding air, a textile detergent or softener, and a product for the upkeep of or for purifying the surrounding air.

17. The composition according to claim 11, wherein, said 5,5-dimethyl-3-ethyl-3,4-dihydrofuran-2-one masks a taste or a smell of said composition.

18. The composition according to claim 11, wherein said 5,5-dimethyl-3-ethyl-3,4-dihydrofuran-2-one is in combination with ingredients selected from the group consisting of at least one flavoring or scenting ingredient, at least one solvent, at least one adjuvant and combinations thereof.

19. The composition according to claim 11, wherein said 5,5-dimethyl-3-ethyl-3,4-dihydrofuran-2-one is present in a concentration of from 0.001% to 99% by weight of said composition.

20. The composition according to claim 19, wherein said 5,5-dimethyl-3-ethyl-3,4-dihydrofuran-2-one is present in a concentration of from 0.001% to 20% by weight of said composition.

21. The composition according to claim 20, wherein said 5,5-dimethyl-3-ethyl-3,4-dihydrofuran-2-one is present in a concentration of from 0.1% to 10% by weight of said composition.

22. A process of synthesizing 5,5-dimethyl-3-ethyl-3,4-dihydrofuran-2-one, comprising the steps of:
reacting sodium methoxide and dimethylmalonate with 3-chloro-2-methylprop-1-ene to form a first product;
reacting said first product with potassium t-butoxide and ethyl bromide to form a second product; and
reacting said second product with sodium hydroxide and phosphoric acid to form 5,5-dimethyl-3-ethyl-3,4-dihydrofuran-2-one.

23. A hair hygiene or care product, comprising 5,5-dimethyl-3-ethyl-3,4-dihydrofuran-2-one.
